(19) United States
(12) Patent Application Publication
(10) Pub. No.: US 2010/0036364 A1
(43) Pub. Date: Feb. 11, 2010

(54) Catheter Introducer

(75) Inventor: Martin Wübbeling, Mannheim (DE)

Correspondence Address: Rutan & Tucker, LLP., 611 ANTON BLVD, SUITE 1400, COSTA MESA, CA, 92626, US

(73) Assignee: C. R. Bard, Inc., Murray Hill, NJ (US)

(21) Appl. No.: 12/536,260

(22) Filed: Aug. 5, 2009

Related U.S. Application Data
(60) Provisional application No. 61/086,628, filed on Aug. 6, 2008.
(60) Provisional application No. 61/227,337, filed on Jul. 21, 2009.

(30) Foreign Application Priority Data
Aug. 6, 2008 (GB) 0814436.2
Jul. 21, 2009 (GB) 0912665.7

(51) Int. Cl. A61M 25/00

(52) U.S. Cl. 604/528

(57) ABSTRACT

A catheter introducer includes a flexible introducer tube that extends percutaneously in use and defines a bore along which a distal shaft portion of a catheter may be advanced into a bodily lumen. The flexible introducer tube includes a co-axial assembly of a structural element having a multiplicity of full wall thickness openings, and a sealing element that restrains fluid flow through the openings, a radial wall thickness of the sealing element being less than a radial wall thickness of the structural element.

### PRIORITY

This application claims the priority benefit of U.S. Provisional Application No. 61/086,628, filed Aug. 6, 2008, U.K. Patent Application No. GB 08 14436.2, filed Aug. 6, 2008, U.S. Provisional Application No. 61/227,337, filed Jul. 21, 2009, and U.K. Patent Application No. GB 09 12665.7, filed Jul. 21, 2009. This application expressly incorporates by reference the entirety of each of the above-mentioned applications as if fully set forth herein.

### TECHNICAL FIELD

This invention relates to a catheter introducer that includes a flexible introducer tube that will in use extend percutaneously through the skin of the patient in order to enable the introduction of a catheter into a bodily lumen.

### BACKGROUND

Catheter introducers are known, with a flexible introducer tube for advancing the catheter into the bodily lumen. A conventional diameter for the bore of the tube is 6.5 French (1 mm=3 F) but other diameters, in a range of from 4 F to 12 F, are commonly available. An introducer with a 6.5 F bore typically exhibits an outside diameter of the introducer tube of the order of 8 French.

Catheters and catheter introducers are navigated by a combination of force applied endwise to the device to longitudinally direct the movement of the device and torque applied about the axis of the catheter to rotate the catheter. It is of importance that the operator have good control over longitudinal and rotational motion. It is simultaneously of importance that the device be sufficiently flexible to avoid the imposition of undue stress on the cavities through which the catheter passes. Catheter introducers, therefore, should provide a pathway with good structural integrity and resistance to applied internal forces while presenting an external configuration that will avoid harm to body passages. Further, the catheter introducer should enable an operator to cleanly and responsively navigate the introducer to the desired location, and should thus provide good tactile feedback to the operator as to obstructions and ease of travel.

Medical practitioners (and their patients) would be interested in a catheter introducer in which the tube exhibits a thinner wall, with no loss of performance and no loss of lumen diameter. For example, if the outer diameter could be reduced from 8 French to 7 French, there would be a 24% reduction in the area of the puncture wound in the skin of the patient that is caused by the percutaneous passage of the introducer tube of the catheter introducer.

It is an objective of the present invention to offer medical practitioners just such an introducer.

### BRIEF SUMMARY

According to the present invention there is provided a catheter introducer as identified above and in which the introducer tube is a co-axial assembly of a structural element that exhibits a multiplicity of full wall thickness openings, and a sealing element that restrains fluid flow through the openings, the radial wall thickness of the sealing element being less than that of the structural element.

Noteworthy is that resort to a co-axial assembly of a plurality of elements can achieve the object of the invention, to reduce the size of the puncture wound.

It is contemplated that the structural element will be a seamless tube, but it could be a seamed tube or even a member that has the general form of a tube yet extends in circumference through an angle of less than 360°. One way to make such a structural element might be to roll flat sheet material into tubular form but presently prepared is a seamless stainless steel tube. Nickel-titanium shape memory alloy is an attractive material, given its quasi super plastic behaviour in deformation, as evidenced by the popularity of NITINOL stents. However, shape memory alloy is relatively expensive compared with stainless steel, and this might be a contra-indication for catheter introducer tube usage where ultimate performance in resilient deformation is not an absolute priority.

The openings in the wall thickness of the structural element have the purpose of endowing the structural element with the required degree of flexibility, and any shape and distribution of the openings is acceptable if it delivers that flexibility. However, the presently preferred form of opening is a slit, having a width that is relatively narrow, such as 20 μm, enabling it to be formed by one pass of a laser cutting apparatus. The laser vaporises the material that was previously in the location of the laser-formed slit. In this way, there is no extra process step, of removing scrap portions of material cut away when the laser cutter advances around the circumference of a large area opening. Since the wall thickness of the structural element, in the radial direction, is contemplated to be of the order of 60 μm, laser cutting of it is not a new engineering problem.

For use as the sealing element, the preferred option at this time is a polyethylene terephthalate tube shrink-fitted to the outside of the structural element. However, overmolding is an alternative to shrink fitting, and meltable polymers with which an overmolding step might be practicable include a polyamide, a polyurethane, or PEBAX. A shrink tube of PTFE-FEP is one alternative to a PET shrink tube.

As to slit patterns, it is presently preferred to arrange each slit strictly perpendicular to the length of the introducer tube and extending more or less all the way around the circumference of the introducer tube except for a short unslitted portion between the facing ends of the slit, the unslitted portion therefore having to carry all of the endwise stresses imposed on the introducer tube. Depending on the design brief, it may be better in terms of flexibility to put a plurality of co-linear slits into each circumference of the introducer tube, so that there are two or more unslitted portions of material to carry the endwise stress. Regardless how many slits are provided in each circumference, the likely optimal arrangement of unslitted material is going to be progressive staggering of the unslit portions, circumferentially, as in a helical arrangement, along the length of the tube. To allow flexibility without preference for flex direction, it is presently preferred to stagger the unslit portions by 90 degrees in a corkscrew (clockwise or anticlockwise) sense along the length of the tube, forming a helix of slits. Other angles, such as angles in the range 10 degrees to 90 degrees, and more preferably angles in the range 30 degrees to 90 degrees, and even more preferably 45 degrees to 90 degrees, may be employed. Additionally, angles greater than 90 degrees might be selected for some applications.

In many applications it will be preferred to provide cuts which extend significantly more than 180 degrees around the circumference, that is, a configuration where the circumferential extent of the slit is much greater than half the circumference. Such a configuration generally provides a good balance of flexibility and endwise strength. However, in such a configuration, under pressure during use the slits may partially close, even to the point where the portion of the slit diametrically opposed to the unslitted portion. This effect can lead to distorted transmission of force sensation through the introducer, which may in turn decrease the ability of the operator to accurately sense obstructions and cleanly navigate the introducer.

To mitigate this effect, the introducer may be provided with the sealing element in such a configuration as to pre-compress the tube; that is, the slits are closed to some desired degree under longitudinal compression provided by tension in the sealing element. Such an effect may be engendered by pre-compressing the tube prior to applying the sealing element, and is particularly advantageously engendered by applying a shrink-fit coating to the tube while the tube is in a state of pre-compression. The choice of coating used and amount of pre-compression applied will affect the degree of pre-compression achieved in the system as a whole, as well as the degree of flexibility retained.

### BRIEF DESCRIPTION OF THE DRAWINGS

For a better understanding of the invention, and to show more clearly how the same may be carried into effect, reference will now be made, by way of example, to the accompanying drawings, in which:

FIG. 1 is a view, from the side, of a preferred embodiment of catheter introducer according to the present invention;

FIG. 2 is a side view of the FIG. 1 introducer, 90° displaced from that of FIG. 1;

FIG. 3 is a detail, to show the distal tip of the FIG. 2 introducer at a larger scale;

FIG. 4 is a view of a portion of the percutaneous tube of FIG. 2, within the circle marked IV in FIG. 2;

FIG. 5a is a view of a first slit pattern used in an embodiment of the present invention;

FIG. 5b is a view of a second slit pattern used in an embodiment of the present invention;

FIG. 6a is a view of a single slit in the pattern of FIG. 5a in an uncompressed state; and

FIG. 6b is a view of a single slit in the pattern of FIG. 5a in a compressed state, under longitudinal compression.

### DETAILED DESCRIPTION

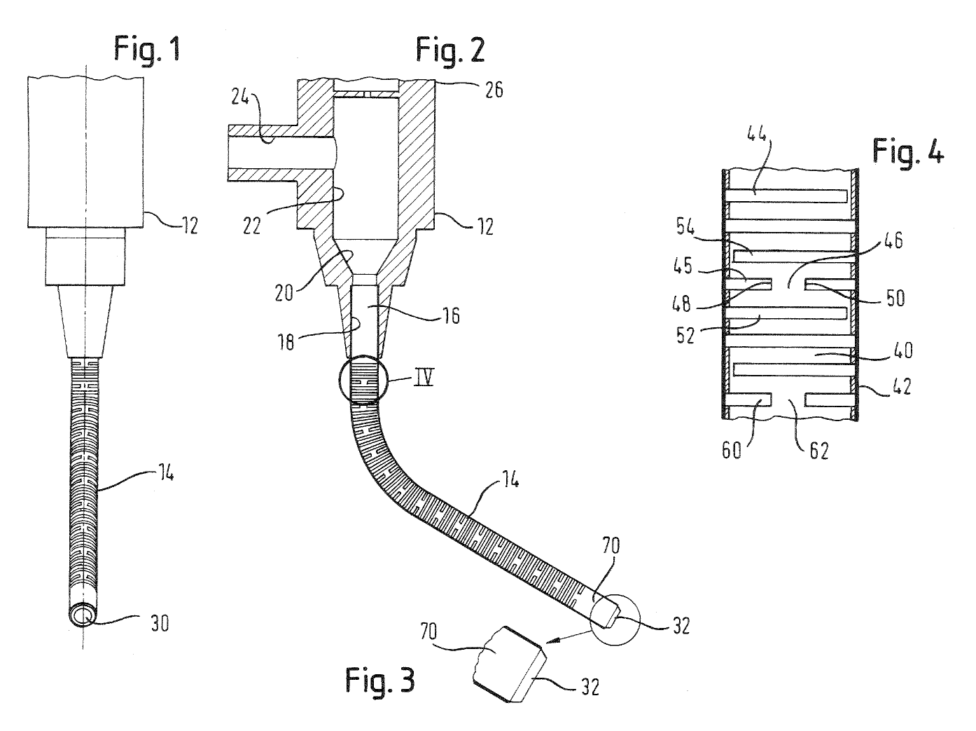

The catheter introducer shown in the FIGS. 1 to 4 has a hub 12 that remains outside the body at all times, and a percutaneous introducer tube 14 that has a proximal end 16 that is received within a bore 18 at the distal end of the hub 12. The bore 18 widens through a taper portion 20 into a larger bore portion 22 with a side lumen 24, all of which is conventional. The distal tip of a catheter can be introduced into the hub 12, from its proximal end 26, advancing along the bore 22 and down the tapered portion 20 to be guided into the bore 30 of the introducer tube 14, at the proximal end 16 of the tube. Thereafter, the distal tip is advanced along the full length of the bore 30 of the introducer tube 14 until it exits the distal tip 32 of the introducer tube and advances onward into the bodily lumen, for which the tube 14 is a catheter introducer.

Looking at the details of construction of the introducer tube 14, it features a stainless steel seamless tube 40 sleeved in a shrink-fitted PET tube 42. Flexibility of the metal/polymer co-axial combination introducer tube is accomplished by a multiplicity of slits 44, cut with a laser through the full wall thickness of the steel tube 40. In this preferred embodiment, the laser cut slits have a width of 20 μm, and the PET shrink fit sealing element has a wall thickness of 20 μm. The wall thickness of the stainless steel tube in this preferred embodiment is 60 μm. It hardly needs stating, that the skilled reader will tailor wall thicknesses and cut widths to the optimal values for the design requirements of the particular introducer tube being manufactured. Electrochemical polishing techniques may conventionally be used after cutting is completed to ensure that the slitted tube has a sufficiently smooth surface for the contemplated application.

Generally preferred is that the tube is prepared in an annealed condition, in order to achieve an enhanced balance of the mechanical properties needed in use, including trackability, pushability, bendability and resistance to kinking and buckling, as well as capability to effectively transmit a torque along the length of the tube.

The diameter of the lumen of the stainless steel tube 40 is of the order of 6.5 French (the unit of length“F”, for“French”, is convenient in this technical field. It signifies ⅓ mm). A 6.5 F bore for a catheter introducer tube is conventional but, in conventional introducer tubes, the resulting outside diameter of the introducer tube is of the order of 8 F. With the present invention, however, in this preferred embodiment, the outside diameter is of the order of 7.0 F. Readers will appreciate that the gain, in moving from an 8 French to a 7 French puncture hole is a reduction of 24% in the cross-sectional area of the puncture hole—a matter of some serious significance for the patient, and for those performing the catheterisation procedure.

Readers will appreciate that the precise pattern of openings in the wall thickness of the structural element of the introducer tube is also a matter of judgement and optimisation. Preferred by the present inventor is to provide the openings as slits that exhibit a length direction strictly perpendicular to the length direction of the introducer tube, each slit extending around an arc of the circumference of the introducer tube that is just short of a full circumference, thereby leaving unslitted material 46 between two facing ends 48, 50 of the particular slit 45. As we see in FIG. 4, the slits 52 and 54, axially adjacent to the slit 45 with ends 48 and 50, have facing ends arranged at other parts of the circumference of the tube. In fact, there is a stagger of 90°, between the ends of a particular slit, and those of the axially next adjacent slit on the introducer tube. This has the consequence that, moving along the length of the tube, the pattern of staggering of the slit ends repeats itself every fourth slit, so that slit 60 has ends facing across unslitted material 62 which lies on the same diameter of the introducer tube 14 as the unslitted material 46 between the ends 48, 50 of slit 45.

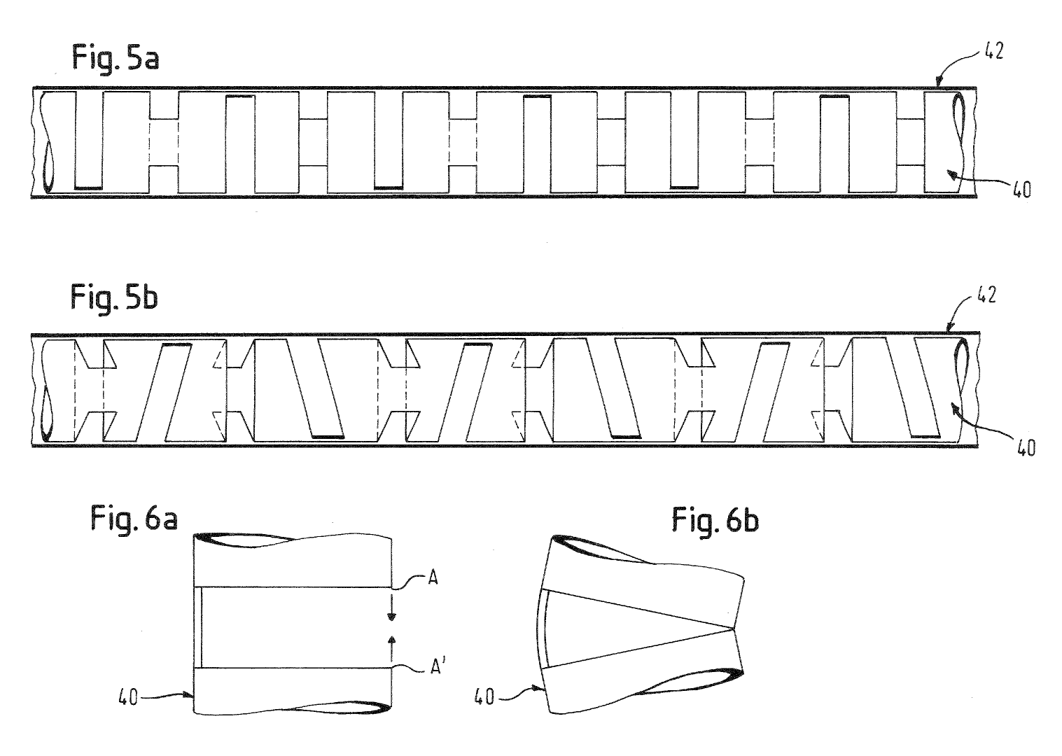

While the structure of the preferred embodiment is shown in detail in FIG. 5a, other embodiments may have slits set at angles other than at 90 degrees to the local tube axis, and have other angular and spatial relationships between neighbouring slits. FIG. 5b shows one alternative embodiment having slits inclined to the plane defined by the local axis of the tube with a helix angle of 90 degrees and equal spacing between the intersections of the slits with the diameter of the tube. The angle of inclination of the slits is exaggerated in FIG. 5b to demonstrate the alternative to the embodiment of FIG. 5a. In the embodiment of FIG. 5b the slits are inclined in the same longitudinal direction from apex to chord (defined by the ends of the slit), enabling the tube to be flexed in all directions equally. The slits, however, need not be cut in a plane, and embodiments having helically-curved slits are also envisioned.

Variation is possible in the depth of the cut of the slits. Provided that the slit is cut more than half-way through the tube, so that the extent of the slit in the circumferential direction exceeds half of the circumference of the tube, the advantages of the present invention may be realised. Cutting to greater depths increases flexibility while reducing columnar strength. Cutting the slits leaves at least one so-called“bridge” of tube material adjoining neighboring tubular rings. Of course, cuts may be made in the bridge to subdivide it, if enhanced flexibility at the cost of columnar strength is required.

Variation is also possible in the width of the cut of the slits, and in the longitudinal pitch of the slits. Reducing the pitch will tend to increase flexibility, and vice versa. Any or all of these parameters may be varied along the length of the tube, in order to impart different strength and flexibility to different sections of the tube. Such longitudinal variation may be of importance to a designer who is seeking a highly flexible distal portion for ease of navigation within the body, while requiring a proximal portion having high columnar strength. Selection of such parameters by trial and experiment is well within the ambit of the skilled person in the art. Further, the slits may indeed have parallel walls, or may, for example, be tapered with the depth of cut.

Turning now to the design details of the distal end of the introducer tube, a chamfered distal end annulus 32 to the tube is evident from FIG. 3. A tip zone 70 immediately proximal of the end annulus has a length of 3mm to 5mm which is free from the slits 44, there being no need for enhanced flexibility in this last short portion of the length of the introducer tube. Because the tip zone is slit-free, there is also no need for the sealing element to extend all the way to the chamfered end annulus of the tube. The PET tube 42 terminates in the tip zone, distal of the distalmost slit 44.

This particular pattern of slits delivers good flexibility but other slit patterns will provide comparable degrees of flexibility. The slits need not be perpendicular to the length direction of the introducer tube but can be slanted to that length direction, allowing the possibility of helical patterns of slits along the length of the introducer tube. One advantage of the perpendicular arrangement over the helical arrangement is that patterns of stress in the introducer tube will be similar, regardless whether the hub 12 is rotated clockwise or anti-clockwise, during the advancement of the introducer tube 14, percutaneously, into its in-use position.

With slits perpendicular to the tube length, one convenient pitch (axial length between centres of axially adjacent slits) is 60 μm. Again, those skilled in the art will optimise this dimension, to meet their specific design requirements.

If pre-compression is desired, before shrinking the sleeve the introducer tube is mechanically compressed endwise within the sleeve while located, for example, on a mandrel. The mandrel is sized to accommodate the introducer tube on it, to ensure that the introducer tube may be compressed linearly without distorting or buckling under the applied endwise compressive force. The force may then be increased until a desired degree of compression is reached, for example to the point where the apex of each slit, the furthest point from the unslitted portion, closes. This change of state is shown in FIG. 6a and 6b; in FIG. 6a a slit is shown in the open, unprecompressed state, while in FIG. 6b the slit is compressed to close the apex of the slit, resulting in the coming together of points A and A′. Other embodiments having different degrees of closure can result from the application of different magnitudes of force, and the force required to achieve a different degree of closure will of course vary with the length of tube. It is entirely within the abmit of the skilled person to make such adjustments to meet the requirements of any use to which the force-transmitting element is put.

In the present embodiment, heat is then applied to the sleeve to cause it to shrink, and thus to snugly enclose and radially compress the already longitudinally-compressed introducer tube. In the present embodiment, heat-shrinking is carried out at a temperature of 200° C.

The sleeve is chosen such that the degree of radial compression achieved is sufficient to frictionally prevent the introducer tube from longitudinally expanding once the external endwise force is released. It is at this stage of the process that, in other embodiments not already having a sleeve already fitted, a retaining coating may be applied, for example by spraying a polymeric coating.

In similar embodiments, the sleeve may be chosen so that the ends of the sleeve enclose the ends of the introducer tube, thereby further constraining its expansion. If the compressive frictional interaction is sufficiently strong, however, such end-capping is not strictly necessary. In other embodiments, the sleeve may be applied later by coating, lamination or moulding, rather than by heat-shrinking. It is sufficient to realise the invention that the sleeve be able to maintain structural integrity while applying a longitudinally compressive force to the introducer tube. In some embodiments, application of adhesive to or roughening of either the sleeve inner surface or tube outer surface, or both, may be used to increase the ability of the sleeve to maintain the required degree of compression in the introducer tube.

Finally, the endwise compressive force is released; the introducer tube is now held in a compressed state by the presence of the sleeve.

The introducer thus formed is resistant to longitudinal compression, as the slits are closed at their apices and thus will not easily close further. However, the introducer may easily be flexed, as deviation from a straight-tube configuration may be achieved by virtue of the slits on one or other side of the tube opening under flexion. The sleeve selected has sufficient resilience to permit this extension of one side or another, while tending to keep the structure as a whole under the longitudinal compression required.

The exact amount of flexibility is likely to be one of those design parameters where the (not necessarily rational) subjective preference of individual doctors is decisive. Readers will grasp that another advantage of the present invention is that the laser cutting regime and pre-compression achieved can easily be tailored to the slit pattern which delivers just that degree of flexibility that the doctor ordered.

The illustrated embodiment is presently a preferred one but other embodiments will likely also work well, and are to be seen as within the scope of the claims which follow. All publications and patent applications cited in this specification are herein incorporated by reference in their entirety as if each individual publication or patent application were specifically and individually put forth herein.

What is claimed is:

1. A catheter introducer, comprising:
a flexible introducer tube that extends percutaneously in use and defines a bore along which a distal shaft portion of a catheter may be advanced into a bodily lumen, the flexible introducer tube including a co-axial assembly of a structural element having a multiplicity of full wall thickness openings, and a sealing element that restrains fluid flow through the openings, a radial wall thickness of the sealing element being less than a radial wall thickness of the structural element.

2. The introducer according to claim 1, wherein the structural element is a seamless tube.

3. The introducer according to claim 1, wherein the openings are slits.

4. The introducer according to claim 3, wherein the slits are parallel to each other.

5. The introducer according to claim 4, wherein each of the slits extend at least halfway around the circumference of the tube.

6. The introducer according to claim 5, wherein the slits are arranged in a helix along the length of the structural element.

7. The introducer according to claim 6, wherein the slits extend around an arc in the circumference of the structural element, the arc forming a perpendicular angle with respect to a longitudinal axis of the structural element.

8. The introducer according to claims 7, wherein slits axially adjacent in the helix are staggered by rotation through a defined angle about the structural element.

9. The introducer according to claim 8, wherein the defined angle about the structural element is 90 degrees.

10. The introducer according to claim 3, wherein the slits have a width of about 20 μm.

11. The introducer according to claim 1, wherein the diameter of the bore of the co-axial assembly is about 6.5 French.

12. The introducer according to claim 1, wherein the outer diameter of the co-axial assembly is about 7.0 French.

13. The introducer according to claim 1, wherein the structural element wall thickness is about 60 μm.

14. The introducer according to claim 1, wherein the sealing element is tubular, and wherein the sealing element wall thickness is about 20 μm.

15. The introducer according to claim 1, wherein the sealing element lies radially outside the structural element.

16. The introducer according to claim 1, wherein the sealing element is an overmolded element.

17. The introducer according to claim 1, wherein the sealing element comprises polyethylene terephthalate (PET).

18. The introducer according to claim 1, wherein the sealing element is subject to no external endwise compressive forces, the structural element being retained in a state of endwise compression by the sealing element such that the slits are relatively more closed than if the sleeve were absent.

19. A catheter introducer, comprising:
a tubular structural element comprised of a metal material and including a plurality of slits imparting flexibility thereto, the slits laser cut through a wall thickness of the structural element generally perpendicular to a longitudinal axis thereof, the slits extending about a circumference of the structural element less than 180 degrees, adjacent slits having rotationally offset ends; and
a tubular sealing element coaxial with the structural element, the sealing element restraining fluid flow through the slits and having a wall thickness less than the wall thickness of the structural element.

20. The introducer according to claim 19, wherein the structural element is retained in a state of endwise compression by the sealing element, the compression acting to at least partially close the slits.
